(19) United States
(12) Patent Application Publication
(10) Pub. No.: US 2026/0256867 A1
(43) Pub. Date: Sep. 3, 2026

(54) COMPOSITION COMPRISING THERAPEUTICALLY EFFECTIVE PHYTOINGREDIENTS AND PROCESS OF ISOLATION THEREOF

(71) Applicant: Kodagikere Palakshappa PRASANNA, Shimoga (IN)

(72) Inventor: Kodagikere Palakshappa PRASANNA, Shimoga (IN)

(21) Appl. No.: 18/854,847

(22) PCT Filed: Apr. 10, 2023
(86) PCT No.: PCT/IN2023/050349

§ 371 Date: Oct. 7, 2024

(30) Foreign Application Priority Data
Apr. 8, 2022 (IN) 202141051137

(51) Int. Cl. A61K 36/9066; A61P 35/00

(52) U.S. Cl. CPC A61K 36/9066; A61P 35/00; A61K 2236/35

(57) ABSTRACT

The present invention generally relates to a composition comprising therapeutically effective phytoingredients. More particularly the present invention relates to a composition comprising therapeutically effective phytoingredients obtained from Curcumin Removed Turmeric Oil (CRTO) and the process of isolation thereof. The present invention discloses a composition comprising therapeutically effective phytoingredients such as Hydroxycurcumin and its analogues obtained from Curcumin Removed Turmeric Oil (CRTO).

### FIELD OF THE INVENTION

The present invention generally relates to a composition comprising therapeutically effective phytoingredients. More particularly the present invention relates to a composition comprising therapeutically effective phytoingredients obtained from Curcumin Removed Turmeric Oil. The composition of the present invention comprises ingredients such as Hydroxycurcumin and its analogues obtained from Curcumin Removed Turmeric Oil (CRTO). The present invention also provides a process of isolation of the said composition from Curcumin Removed Turmeric Oil (CRTO).

### BACKGROUND OF THE INVENTION

Turmeric Rhizome, the humble condiment has social, cultural and spiritual significance in many parts of the world. India is the world leader in the cultivation of Turmeric accounting for 78% followed by China at 8%, Myanmar at 4% other countries including Bangladesh, Nigeria, Indonesia and Vietnam bring up the rest 6%.

The exact geographic origin of turmeric is unknown, but it is a safe bet that it could be in Southeast Asia (Velayudhan et al. 1999). Watt (1972), reported that there is no conclusive evidence to show that Curcuma longa is a native of India, though several species of Curcuma are found in India. The greatest diversity of turmeric species is found in India, Myanmar, and Thailand.

Turmeric, Haridra, (Curcuma longa Linn.) is a widely used shrub in folk and Ayurvedic systems of medicine. Haridra, is prescribed in the form of Lepa, Kashaya, Kshara, Churna, Taila, Ghrita, Peya, Kwatha and Vasti in various diseases (Prameha, Kushtha, Shotha etc.) in different Samhitas and Chikitsa Granthas. The therapeutic uses of Haridra are described in different Samhita and Chikitsa Grantha in different ailments like Kushtha (Skin disease), Prameha (Diabetes Mellitus), Jwara (Fever), Vrana (Wound), Arsha (Piles), Pandu (Anaemia), Kamala (Jaundice), Vatarakta (Gaute), Hikka-Shwasa (respiratory diseases), Visha (Poisoning), Shotha (Oedema) and Mukharoga (Oral cavity disorder) etc. It is reported to be anti-inflammatory, antidiabetic, hypocholesterolemic, chemo-preventive agent, stomachic, tonic, blood purifier, anthelmintic, antacid, carminative, skin affections, soothing inflamed joints, curative properties for affection of the liver and gall bladder.

As it would be evident, turmeric has been extensively used to address and treat various ailments of the body in Ayurveda and continues its status as the “golden spice” to date.

In modern times, the interest in turmeric started out as a culinary spice that imparts the bright yellow color and the flavor to food. It was also used as a dyeing agent to impart it's brilliant yellow unto clothes. The Buddhist monks traditionally dyed their robes yellow with turmeric as an embodiment of Bhoddisattva Rathnasambhava who was associated with the color yellow.

With the advent of industrial practices and research, this color was attributed to its content of Curcumin, one of the three curcuminoids that gave turmeric its yellow color. A study into this “color” to elicit any medicinal properties introduced curcuminoids as the three Curcumins are collectively called, into modern therapeutic practice. The curcuminoids were found to be a potent antioxidant and anti-inflammatory which led to its interest in supplements and nutraceuticals alike.

Chemical constituents of various tissues of turmeric (Curcuma longa L.) have been extensively investigated. To date, at least 235 compounds, primarily phenolic compounds and terpenoids have been identified from the species, including 22 diarylheptanoids and diarylpentanoids, eight phenylpropene and other phenolic compounds, 68 monoterpenes, 109 sesquiterpenes, five diterpenes, three triterpenoids, four sterols, two alkaloids, and 14 other compounds. Curcuminoids (diarylheptanoids) and essential oils are major bioactive ingredients showing various bioactivities in in-vitro and in-vivo bioassays. Curcuminoids in turmeric are primarily accumulated in rhizomes. The essential oils from leaves and flowers are usually dominated by monoterpenes while those from roots and rhizomes primarily contained sesquiterpenes. The contents of curcuminoids in turmeric rhizomes vary often with varieties, locations, sources, and cultivation conditions, while there are significant variations in composition of essential oils of turmeric rhizomes with varieties and geographical locations.

### Structure of Few Active Compounds in Turmeric:

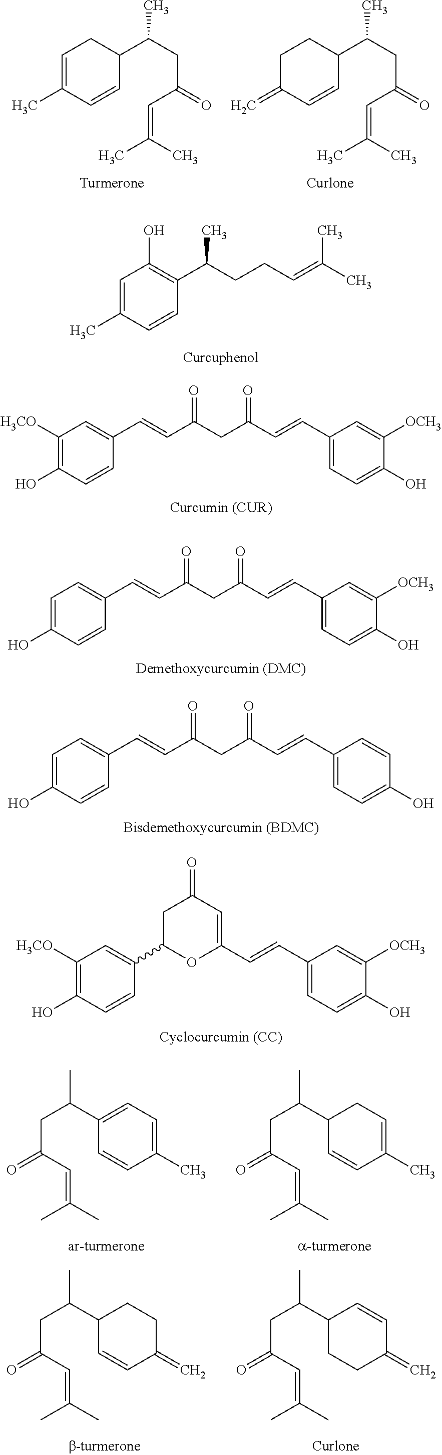

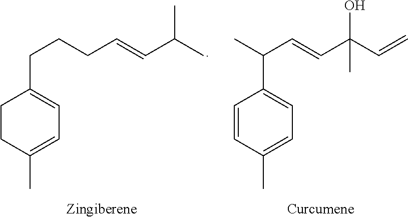

Of 110 species of the genus Curcuma, only about 20 species have been studied phytochemically (Nahar L, 2007). Curcuma longa is the most chemically investigated species of Curcuma. To date, at least 235 compounds, primarily phenolic compounds and terpenoids have been identified, including diarylheptanoids (including commonly known as curcuminoids), diarylpentanoids, monoterpenes, sesquiterpenes, diterpenes, triterpenoids, alkaloids, and sterols, etc.

Commercial Curcumin is usually a mixture of three curcuminoids. The curcuminoids are present in amounts as Curcumin (70-80%), Demethoxycurcumin (15-25%) and Bisdemethoxycurcumin (2.5-6.5%). The studies on curcuminoids have constantly maintained that the specific ratio of the three curcuminoids is primarily the reason for its therapeutic efficacy.

Hydroxycurcumin, better known as Bisdemethoxycurcumin is one of the active component of the turmeric species and has been consumed in food and for medicinal purposes for thousands of years. Extensive research has shown that, Hydroxycurcumin modulates various signalling molecules, including inflammatory molecules, transcription factors, enzymes, antitumor functions, such as proliferation inhibition, metastasis suppression and apoptosis induction, in many cancer types. Hydroxycurcumin is also known to be a potent antioxidant.

Biological activity of Curcumin metabolites has demonstrated low plasma levels of parent compound which is the reason for lack of complete translation of experimental findings to clinical practices. Evidence show that the analogue Hydroxycurcumin possesses promising biological and pharmacological effects. Stable analogues like Hydroxycurcumin (Bisdemethoxycurcumin) by virtue of its increased stability, show improved nuclear cellular uptakes compared to curcuminoids. Hydroxycurcumin analogues inhibit proliferation and survival of several types of cancer cells, which includes breast, colon, leukaemia, glioma, lung and liver cancer.

Generally, curcuminoids include Curcumin, Demethoxycurcumin and Bisdemethoxycurcumin, extracted from Turmeric rhizome. The composition of the three are 70, 20 and 10% respectively. Curcuminoids have a higher content of methoxy groups, known to be hydrophobic, and less hydroxy groups, more hydrophilic in nature. Curcuminoids have been found to have poor water solubility and therefore has limited applications in various water-based formulations. Bisdemethoxycurcumin (Hydroxycurcumin), by virtue of its hydroxyl groups, is far more polar soluble.

Hydroxycurcumin complex (HCC) has a wide range of bioactivity as, antioxidant, anti-aging, anti-inflammatory, anti-tumor, anti-cancer, anti-mutagenic, hepatoprotective, cell proliferation, cardioprotective, gastroprotective, anti-thrombotic and anti-microbial activities. Stable analogues like Hydroxycurcumin may, by virtue of its increased stability, show improved nuclear cellular uptakes compared to curcuminoids. Hydroxycurcumin analogues inhibit proliferation and survival of several types of cancer cells, which includes breast, colon, leukaemia, glioma, lung and liver cancer.

The industrial manufacturing of curcuminoids has fairly remained unchanged for over six decades. One of the stumbling blocks that the industry faces is that the extraction and isolation process of curcuminoids has low efficiency, on average, of 50-60%. Hence, it may be inferred that a significant content of curcuminoids remains trapped in CRTO (Curcumin Removed Turmeric Oil). Only 1.8-2.2% of the total weight of raw materials is what is isolated as finished product and the rest is either discarded as marc and CRTO.

With an herb as versatile as turmeric, as is evidenced by its extensive use in various forms and formats in Ayurveda, isolating a measly 1.8-2.2% from this herb and discarding the rest is a waste of natural resources. The CRTO has always been a complex by-product containing several phytochemical ingredients which has constantly baffled scientists and industrialists alike. Today, apart from some attempts in isolating turmeric oil from CRTO, it is mostly used as cheap fuel, burning up all the goodness of its phytoingredients.

It is therefore an objective of the present invention to identify and isolate a composition comprising therapeutically effective phytoingredients from Curcumin Removed Turmeric Oil.

### SUMMARY OF INVENTION

The present invention generally relates to a composition comprising therapeutically effective phytoingredients. More particularly, the present invention relates to a composition comprising therapeutically effective phytoingredients obtained from the Curcumin Removed Turmeric Oil (CRTO). The present invention also provides for a process for isolation of the composition comprising therapeutically effective phytoingredients such as Hydroxycurcuminoids and its analogues in higher amounts from CRTO.

According to an embodiment of the invention there is provided a composition comprising therapeutically effective phytoingredients obtained from the Curcumin Removed Turmeric Oil (CRTO).

According to an embodiment of the invention the composition of the present invention comprises of major curcuminoids such as Hydroxycurcumin (Bisdemethoxycurcumin), Demethoxycurcumin, Curcumin, and their analogues.

According to an embodiment of the invention the composition of the present invention comprises Hydroxycurcumin (Bisdemethoxycurcumin) in the range of 6-27%, Demethoxycurcumin in the range of 9-13%, Curcumin in the range of 2-4% and other phenolic compounds.

In an embodiment of the invention, the composition of the present invention is also referred to as the Hydroxycurcumin complex (HCC). The Hydroxycurcumin complex with higher content of hydroxyl groups and minimal methoxy groups makes the complex more stable and more bioavailable in contrast to Curcuminoids that has a higher content of methoxy groups, known to be hydrophobic, and less hydroxy groups, more hydrophilic in nature.

According to an embodiment of the invention the composition of the present invention is effective in protecting the liver against oxidative stress as has been investigated through in-vitro studies by the inventor.

According to another embodiment of the invention the composition of the present invention shows anti-cancer activity as has been investigated through in-vitro studies by the inventor.

According to an embodiment of the invention the composition of the present invention is safe and is free of any adverse effects and the same is established by toxicity study.

According to another embodiment of the invention the composition of the present invention may also be formulated into suitable dosage forms selected from a group comprising of powder, paste, tablets, syrups and/or capsules, soft gels or any other suitable formulation.

According to an embodiment of the invention there is provided a process that utilizes the waste matter, industrially known as Curcumin Removed Turmeric Oil (CRTO) which is discarded after extracting curcuminoids. The CRTO “mother liquor” is still a homogeneous composition with the curcuminoids still present but contaminated with other resinous compounds. Thus, the inventor of the present invention has been able to isolate a composition comprising therapeutically effective phytoingredients from CRTO. The process comprises the steps of:
a) removing the oil content of the Curcumin Removed Turmeric Oil by mixing the CRTO with a non-polar solvent wherein the ratio of the amounts of CRTO and the solvent used is 1:3 to 1:6 weight by volume and heating this mixture for approximately 30 to 120 minutes;
b) filtering the extract and repeating the process 3 more times to effectively remove oil content from the CRTO and obtain a left-over material;
c) pooling all the four extractives and concentrating in a rotary evaporator at a temperature of 40-55° C. in a rotary evaporator to obtain crude turmeric oil with an yield of 40 to 60% w/w wherein the turmeric oil isolated contains not less than 60% of total turmerones including α, β and ar-turmerones;
d) adding a non-polar solvent to the left-over material or spent and heating the solution for about 1 hour wherein the ratio of left over material and non-polar solvent is 1:3 to 1:6 weight by volume;
e) filtering the extract through Whatman's filter paper;
f) repeating this process 7 more times till complete recovery of curcuminoids and pooling all the extractives and concentrating to skip off the solvent;
g) reducing the volume of the extract thus obtained to 1:1 ratio on input and cooling to a temperature of 10-15° C. for 8 hours, obtaining the crystals, filtering and drying in an oven at a temperature of 50-90° C. for 8 hours to obtain curcuminoids powder;
h) drying the spent or left-over material at a temperature of 80° C. under vacuum to remove the solvent and cooling to room temperature to obtain a reddish brown powder which is the composition comprising therapeutically effective phytoingredients;
i) analysing the composition by HPLC for its curcuminoids content.

In an embodiment of the invention, the therapeutically effective phytoingredient composition thus obtained comprises of Bisdemethoxycurcumin (Hydroxycurcumin) in the range of 6-27%, Demethoxycurcumin in the range of 9-13% and Curcumin in the range of 2-4% and other phenolic compounds.

In an embodiment of the invention in the step g) of the aforementioned process, the curcuminoids recovered are 95%.

In an embodiment of the invention in the process to obtain the composition of the present invention, the temperature of heating the mixture of CRTO and non-polar solvent in step a) is preferably 40-55° C. and wherein the mixture is heated for preferably 45 to 60 minutes.

In an embodiment of the invention in the process to obtain the composition of the present invention, the non-polar solvent in step a) is selected from hexane, heptane, pentane, toluene, petroleum ether, diethyl ether or any other suitable solvent.

In an embodiment of the invention in the process to obtain the composition of the present invention, the non-polar solvent taken in step d) may be selected from ethyl acetate, diethyl ether, toluene, heptane, methylene di-chloride or any other suitable non-polar solvent.

In an embodiment of the invention in the process to obtain the composition of the present invention, the ratio of the amounts of CRTO and the solvent used in step a) is preferably 1:3 weight by volume.

In another embodiment of the invention there is provided an alternative process to obtain the composition of the present invention from CRTO wherein the process involved is steam distillation of CRTO at 90-120° C. After steam distillation, water is removed completely, and the left-over material is dissolved in methanol. The solution is then subjected to a temperature of 60-70° C. to remove the methanol completely and a powder is obtained. This powder is then analyzed by HPLC for its curcuminoids content.

In another embodiment of the invention there is provided another process of to obtain the composition of the present invention from CRTO wherein the process involves the steps of:
a) removing the oil content of the Curcumin Removed Turmeric Oil by mixing the CRTO with a non-polar solvent wherein the ratio of the amounts of CRTO and the solvent used is 1:3 to 1:6 weight by volume, preferably 1:3, and wherein the non-polar solvent may be selected from hexane, petroleum ether, heptane, pentane, toluene, diethyl ether or any other suitable solvent;
b) heating the mixture of step a) at a temperature of 40-45° C. for approximately 45 to 60 minutes;
c) filtering the extract after 45 to 60 minutes and repeating the process 3 more times to effectively remove oil content from the CRTO and obtain a left-over material;
d) extracting the left-over material in a polar solvent wherein the solvent may be methanol, ethanol, iso-propyl alcohol or any other suitable solvent wherein the ratio of left over material and the polar solvent is 1:4 to 1:6 weight by volume, preferably 1:4;
e) taking a measured quantity of de-mineralized water in a round bottom (RB) flask and slowly adding the methanol extract to precipitate curcuminoids, filtering, and drying the precipitate at a temperature of 60-85° C.;
f) obtaining the dried powder which is then analyzed by HPLC for its curcuminoids content.

In an embodiment of the invention, the composition isolated by the present invention comprises of major curcuminoids such as Curcumin, Demethoxycurcumin, Hydroxycurcumin and their analogues.

In an embodiment of the invention, the final composition obtained comprises of Bisdemethoxycurcumin (Hydroxycurcumin) in the range of 6-27%, Demethoxycurcumin is in the range of 9-13% and Curcumin in the range of 2-4% and other phenolic compounds.

The above and other features and aspects of the present invention are more clearly described in the complete specification.

### BRIEF DESCRIPTION OF DRAWINGS

The invention can be described in the terms of the following figures where-FIG. 1 illustrates the overlay bar graph showing the % total NO production observed in the untreated control, H₂O2, H₂O₂ and the composition of the present invention (HCC) in various concentrations (6.25, 12.5, 25, 50 and 100 μg/ml) versus the standard control taken of Silymarin at 200 μM/ml.

FIG. 2a shows the cytotoxicity effects of untreated control after the incubation period of 24 hours on A549 cell lines.

FIG. 2b illustrates the cytotoxicity effects of 20 μM/ml concentration of Cisplatin after the incubation period of 24 hours on A549 cell lines.

FIG. 2c shows the cytotoxicity effects of 12.5 μg/ml concentration of composition of the present invention (HCC) after the incubation period of 24 hours on A549 cell lines.

FIG. 2d shows the cytotoxicity effects of 25 μg/ml concentration of composition of the present invention (HCC) after the incubation period of 24 hours on A549 cell lines.

FIG. 2e illustrates the cytotoxicity effects of 50 μg/ml concentration of composition of the present invention (HCC) after the incubation period of 24 hours on A549 cell lines.

FIG. 2f illustrates the cytotoxicity effects of 100 μg/ml concentration of composition of the present invention (HCC) after the incubation period of 24 hours on A549 cell lines.

FIG. 2g shows the cytotoxicity effects of 200 μg/ml concentration of composition of the present invention (HCC) after the incubation period of 24 hours on A549 cell lines.

FIG. 2h shows the cytotoxicity effects of 400 μg/ml concentration of composition of the present invention (HCC)) after the incubation period of 24 hours on A549 cell lines.

FIG. 3 is depicting the overlay bar graph showing the precent cell viability values of A549 cells treated with different concentrations of the composition of the present invention (HCC), vs. controls and standard control after the treatment period of 24 hrs.

### DETAILED DESCRIPTION

Discussed below are some representative embodiments of the present invention. The invention in its broader aspects is not limited to the specific details and representative methods. The illustrative examples are described in this section in connection with the embodiments and methods provided. The invention according to its various aspects is particularly pointed out and distinctly claimed in the attached claims read in view of this specification.

It is to be noted that, as used in the specification and the appended claims, the singular forms “a,” “an,” and “the” include plural referents unless the context clearly dictates otherwise. Thus, for example, reference to a composition containing “a compound” includes a mixture of two or more compounds. It should also be noted that the term “or” is generally employed in its sense including “and/or” unless the content clearly dictates otherwise.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic. Also, references to the term “comprising” may also include the case of “consisting of” and ‘consisting essentially of’ and may be used interchangeably.

The expression of various quantities in terms of “%” means the percentage by weight of the total solution or composition unless otherwise specified.

All cited references are incorporated herein by reference in their entireties. Citation of any reference is not an admission regarding any determination as to its availability as prior art to the claimed invention.

Technical and scientific terms used herein have the meaning commonly understood by one of skill in the art to which the present invention pertains, unless otherwise defined. Reference is made herein to various methodologies and materials known to those of skill in the art.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The present invention, in its product and process aspects, is described in detail as follows.

The present invention relates to a composition obtained from the Curcumin Removed Turmeric Oil (CRTO). More particularly, the present invention relates to a composition comprising therapeutically effective phytoingredients which are obtained from the curcumin removed turmeric oil (CRTO). Also provided is a process for isolation of the composition comprising therapeutically effective phytoingredients such as Hydroxycurcuminoids and its analogues in higher amounts from Curcumin Removed Turmeric Oil (CRTO).

According to an embodiment of the invention there is provided a composition comprising therapeutically effective phytoingredients obtained from the Curcumin Removed Turmeric Oil (CRTO) wherein the composition comprises of major curcuminoids such as Hydroxycurcumin (Bisdemethoxycurcumin), Demethoxycurcumin, Curcumin, and their analogues.

According to an embodiment of the invention the composition of the present invention comprises Bisdemethoxycurcumin (Hydroxycurcumin) in the range of 6-27%, Demethoxycurcumin is in the range of 9-13% and Curcumin in the range of 2-4% and other phenolic compounds.

In an embodiment of the invention, the composition of the present invention is also referred to as the Hydroxycurcumin complex (HCC).

According to an embodiment of the invention the composition of the present invention is effective in protecting the liver against oxidative stress as has been investigated through in-vitro studies by the inventor. The oxidative stress on the liver may be caused due to reasons such as sepsis, hypertension, type II diabetes, hypoxia, cancer etc.

According to another embodiment of the invention the composition of the present invention shows anti-cancer activity as has been investigated through in-vitro studies by the inventor. The Hydroxycurcumin complex showed cytotoxic activity in the in-vitro studies.

According to an embodiment of the invention, the composition of the present invention is effective against cancerous cells. In an embodiment the composition is effective against lung cancer.

According to an embodiment of the invention the composition of the present invention is safe and is free of any adverse effects and the same is established by toxicity study.

According to another embodiment of the invention the composition of the present invention may also be formulated into suitable dosage forms selected from a group comprising of powder, paste, tablets, syrups and/or capsules, softgels or any other suitable formulation.

According to an embodiment of the invention there is provided a process to obtain a composition containing therapeutically effective phytoingredients from Curcumin Removed Turmeric Oil (CRTO) which is generally discarded after extracting curcuminoids. The CRTO “mother liquor” is still a homogeneous composition with the curcuminoids still present but contaminated with other resinous compounds. Thus, the inventor of the present invention has been able to isolate a composition comprising therapeutically effective phytoingredients from CRTO.

In an embodiment of the invention the process comprises the steps of:
a) removing the oil content of the Curcumin Removed Turmeric Oil by mixing the CRTO with a non-polar solvent wherein the ratio of the amounts of CRTO and the solvent used is 1:3 to 1:6, preferably 1:3, weight by volume and heating this mixture for approximately 30 to 120 minutes;
b) filtering the extract and repeating the process 3 more times to effectively remove oil content from the CRTO and obtain a left-over material;
c) pooling all the four extractives and concentrating in a rotary evaporator at a temperature of 40-55° C. in a rotary evaporator to obtain crude turmeric oil with an yield of 40 to 60% w/w wherein the turmeric oil isolated contains not less than 60% of total turmerones including α, β and ar-turmerones;
d) adding a non-polar solvent to the left-over material or spent and heating the solution at a temperature of 50-90° C., preferably 60-70° C. for about 1 hour wherein the ratio of left over material and non-polar solvent is 1:3 to 1:6, preferably 1:3, weight by volume;
e) filtering the extract through Whatman's filter paper;
f) repeating this process 7 more times till complete recovery of curcuminoids and pooling all the extractives and concentrating at a temperature of 50-80° C., preferably 60-70° C. to skip off the solvent;
g) reducing the volume of the extract thus obtained to 1:1 ratio on input and cooling to a temperature of 10-15° C. for 8 hours, obtaining the crystals, filtering and drying in an oven at a temperature of 50-90° C. for 8 hours to obtain 95% curcuminoids powder with a yield of 1.7% to 2%;
h) drying the spent or left-over material at a temperature of 80° C. under vacuum to remove the solvent and cooling to room temperature. This results in reddish brown powder with an yield of 20 to 50 to obtain a reddish brown powder which is the composition comprising therapeutically effective phytoingredients;
i) analysing the composition by HPLC for its curcuminoids content.

The therapeutically effective phytoingredient composition comprises Bisdemethoxycurcumin (Hydroxycurcumin) in the range of 6-27%, Demethoxycurcumin in the range of 9-13% and Curcumin in the range of 2-4% and other phenolic compounds.

In an embodiment of the invention in the process to obtain the composition of the present invention, the temperature of heating the mixture of CRTO and non-polar solvent in step a) is preferably 40-55° C. and wherein the mixture is heated for preferably 45 to 60 minutes.

In an embodiment of the invention in the process to obtain the composition of the present invention, the non-polar solvent in step a) is selected from hexane, toluene, heptane, pentane, petroleum ether, diethyl ether or any other suitable solvent.

In an embodiment of the invention in the process to obtain the composition of the present invention, the non-polar solvent taken in step d) may be selected from ethyl acetate, diethyl ether, toluene, heptane, methylene di-chloride or any other suitable non-polar solvent.

In an embodiment of the invention there is provided an alternative process to obtain the composition of the present invention wherein the process comprises taking CRTO in a 3 neck round bottom flask and subjecting the CRTO to steam distillation at 90-120° C. After steam distillation, water is removed completely and the left-over material is dissolved in a polar solvent such as methanol. The solution is then subjected to a temperature of 60-70° C. to remove the solvent completely and a powder is obtained. This powder is then analyzed by HPLC for its curcuminoids content.

In another embodiment of the invention there is provided another process to obtain the composition of the present invention from CRTO wherein the process involves the steps of:
a) removing the oil content of the Curcumin Removed Turmeric Oil by mixing the CRTO with a non-polar solvent wherein the ratio of the amounts of CRTO and the solvent used is 1:3 to 1:6, preferably 1:3, weight by volume and wherein the non-polar solvent may be selected from hexane, petroleum ether, heptane, pentane, diethyl ether, toluene, or any other suitable solvent;
b) heating this mixture at a temperature of 40-45° C. for approximately 45 to 60 minutes;
c) filtering the extract after 45 to 60 minutes and repeating the process 3 more times to effectively remove oil content from the CRTO and obtain a left-over material;
d) extracting the left over material in a polar solvent wherein the solvent may be methanol, ethanol, iso-propyl alcohol or any other suitable solvent wherein the ratio of left over material and the polar solvent is 1:4 to 1:6, preferably 1:4, weight by volume;
e) taking a measured quantity of de-mineralized water in a round bottom (RB) flask and slowly adding the methanol extract to precipitate curcuminoids, filtering, and drying the precipitate at a temperature of 60-85° C.;
f) obtaining the dried powder which is then analyzed by HPLC for its curcuminoids content.

In an embodiment of the invention the phytoingredient composition isolated by the present invention comprises of major curcuminoids such as Curcumin, Demethoxycurcumin, Hydroxycurcumin and their analogues.

In an embodiment of the invention, the final composition obtained comprises of Bisdemethoxycurcumin (Hydroxycurcumin) in the range of 6-27%, Demethoxycurcumin is in the range of 9-13% and Curcumin in the range of 2-4% and other phenolic compounds.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

In this example, the representative compositions containing therapeutically effective phytoingredients are described. Table 1 shows the amounts of the curcuminoids present in the composition.

TABLE 1
Amount of the curcuminoids in the compositions
of the present invention

| | Bisdemethoxy | Demethoxy | |
|---|---|---|---|
| Actives | curcumin | curcumin | Curcumin |
| Composition 1 | 7.3% | 10.8% | 3.9% |
| Composition 2 | 6.5% | 11.2% | 3.6% |
| Composition 3 | 8.1% | 12.7% | 3.3% |
| Composition 4 | 13.2% | 9.7% | 2.3% |
| Composition 5 | 26.8% | 9.4% | 3.2% |

### Example 2

1 kg of CRTO was taken in a round bottom flask and to it were added 3 volumes of petroleum ether. This mixture was heated gently at a temperature of 40-55° C. for 45 minutes. After 45 minutes, the extract was filtered. This process was repeated 3 more times to effectively remove oil content from the CRTO. All the 4 extractives were pooled and concentrated in a rotary evaporator at a temperature of 40-55° C. Crude turmeric oil was obtained. While the left over material was dissolved in 3 volumes of ethyl acetate. The ethyl acetate from the solution was then skipped off completely by heating the solution at a temperature of 60-70° C. for 1 hour. The extract was filtered through Whatman's filter paper. This process is repeated 7 more times. All the extractives are pooled and concentrated at a temperature of 60-70° C. the volume of extract is reduced to 1:1 ratio on input and cooled to 10-15° C. for 8 hours, crystals are observed, filtered and dried in an oven at 50-90° C. for 8 hours to obtain 95% curcuminoids powder. The spent or left-over material is dried at 80° C. under vacuum to remove the solvent and cooled to room temperature to get reddish brown colored powder. The powder was weighed accurately. The net yield was 25.2% by weight (net weight-252 grams) out of which the contents of the curcuminoids viz. Hydroxycurcumin (Bisdemthoxycurcumin), Demethoxycurcumin and curcumin were 7.3%, 10.8% and 3.9% respectively as determined by HPLC.

### Example 3

1 kg of CRTO was taken into a 3 neck Round bottom flask. This oil content of the CRTO was removed by subjecting CRTO to steam distillation at 90-120° C. After steam distillation water was removed completely. The left-over material was then dissolved in methanol. The methanol from the solution was skipped off completely at 60-70° C. The resultant obtained was a dry powder and it was weighed accurately. The net yield was 27.1% by weight (net weight-271 grams) out of which the contents of the curcuminoids viz. Hydroxycurcumin (Bisdemthoxycurcumin), Demethoxycurcumin and curcumin were 6.5%, 11.2% and 3.6% respectively as determined by HPLC.

### Example 4

1 kg of CRTO was taken in a round bottom flask. To the CRTO were added 3 volumes of hexane. The mixture was heated gently at a temperature of 40-45° C. for 45 minutes. After 45 minutes, the extract was filtered. This process was repeated 3 more times to effectively remove oil content from the CRTO. All the 4 extractives were pooled and concentrated in a rotary evaporator at a temperature of 40-55° C. Crude turmeric oil was obtained. While the left over material was dissolved in minimum volume of a polar solvent such as methanol. 5 litres of De-mineralized water was taken in a round bottom flask and to it was slowly added the methanol extract to precipitate curcuminoids. The precipitate was filtered and dried at 60-85° C. The resultant obtained was a dry powder and it was weighed accurately. The net yield was 26.3% by weight (net weight-263 grams) out of which the contents of the curcuminoids viz. Hydroxycurcumin (Bisdemthoxycurcumin), Demethoxycurcumin and curcumin were 8.1%, 12.7% and 3.3% respectively as determined by HPLC.

### Example 5

1 kg of CRTO was taken in a round bottom flask. To the CRTO were added 3 volumes of hexane. The mixture was heated gently at 40-55° C. for 45 to 60 minutes. After 45 to 60 minutes, the extract was filtered. This process was repeated 3 more times to effectively remove oil content from the CRTO. All the 4 extractives were pooled and concentrated in a rotary evaporator at a temperature of 40-55° C. Crude turmeric oil was obtained. While the left-over material was dissolved in 6 volumes of non-polar solvent. This process was repeated 7 more times. All the extractives were collected and concentrated at a temperature of 60-70° C. The volume of extract is reduced to 1:1 ratio on input and cooled to 10-15° C. for 8 hours, crystals are observed, filtered and dried in an oven at 50-90° C. for 8 hours to obtain 95% curcuminoids powder and the yield was 1.8%. The spent or the left-over material is dried at a temperature of 60-85° C. under vacuum to remove the solvent and cooled to room temperature to get reddish brown colored powder. The resultant obtained was a dry powder and it was weighed accurately. The net yield was 24.3% by weight (net weight-243 grams) out of which the contents of the curcuminoids viz. Hydroxycurcumin (Bisdemthoxycurcumin), Demethoxycurcumin and curcumin were 13.2%, 9.7% and 2.3% respectively as determined by HPLC.

### Example 6

1 kg of CRTO was taken in a round bottom flask. To the CRTO were added 3 volumes of hexane. The mixture was heated gently at 40-45° C. for 45 to 60 minutes. After 45 to 60 minutes, the extract was filtered. This process was repeated 3 more times to effectively remove oil content from the CRTO. The left-over material was dissolved in 6 volumes of non-polar solvent. The step was repeated 7 more times. All the extractives were collected and concentrated at a temperature of 60-70° C. The volume of extract is reduced to 1:1 ratio on input and cooled to 10-15° C. for 8 hours, crystals are observed, filtered and dried in an oven at 50-90° C. for 8 hours to obtain 95% curcuminoids powder and the yield was 1.6%. The left-over material is dried at a temperature of 60-85° C. under vacuum to remove the solvent and cooled to room temperature to get reddish brown colored powder. The resultant obtained was a dry powder and it was weighed accurately. The net yield was 24.3% by weight (net weight-243 grams) out of which the contents of the curcuminoids viz. Hydroxycurcumin (Bisdemthoxycurcumin), Demethoxycurcumin and curcumin were 26.8%, 9.4% and 3.2% respectively as determined by HPLC.

### Example 7

### In-Vitro Study to Assess the Efficacy of the Composition of the Present Invention

### Objective:

The objective of the study was to assess the hepatoprotective effect of the composition of the present invention on HepG2 cell lines. The total nitric oxide inhibition was studied on HepG2 cell lines.

### Background of the Study:

Nitric oxide (NO) is produced from L-Arginine by nitric oxide synthase in biological systems. NO is a pleiotropic biological mediator involved in diverse functions such as vasorelaxation by activating soluble guanyl atecyclase, inhibition of tumour cells and activities ranging from neuronal function to immune system regulation. Altered levels of NO are indicative of sepsis, hypertension, type II diabetes, hypoxia, cancer etc.

NO is a gaseous free radical which has very short half-life in vivo of a few seconds. Therefore, levels of more stable NO metabolites; nitrite (NO2-) and nitrate (NO3-) are detected spectrophotometrically to estimate NO concentration in biological fluids indirectly.

### Principle of the Study:

This assay is based on the reduction of nitrate (NO3-) to nitrite (NO2-) by a reducing agent at 37° C. Converted nitrite and endogenous nitrite are collectively converted by Griess reagent to a blue colored azo compound. This compound can be measured spectrophotometrically between 580-630 nm and absorbance is directly proportional to the total nitric oxide concentration in the sample which is calculated from standard plot.

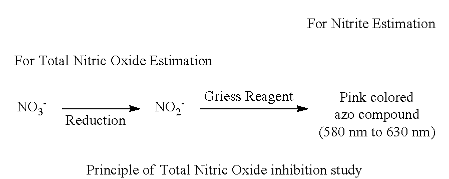

### Cell Line and Culture Medium:

This work was carried out in human liver hepatocarcinoma (HepG2) cells. The HepG2 cells were obtained from National Centre for Cell Science, Pune, India. Cell culture medium: DMEM-Low Glucose-(#AL149, Himedia)

### Assay Controls:

(i) Standard Reagent (Provided in Kit)
(ii) Blank—Culture Medium
(iii) Untreated-Cells cultured media alone
(iv) Toxic controls-Cell cultured in media treated with 100 uM of H2O2
(v) Std control-H₂O₂ induced cells in media with 200 uM/ml of Silymarin

### Procedure:

### Sample Preparation

### Cell Supernatants—

1. Cultured cells upon 70-80% confluence, were seeded in 96 well plate with the density of 20,000 cells/well and incubated for the 24 hours.
2. Afterwards, the spent media was removed and hepatotoxicity induced to the cells with 100 μM/ml of H₂O₂ for 2 hours in all the wells except in Untreated wells and treat cells with the different concentrations (6.25, 12.5, 25, 50 and 100 μg/ml) of the composition of the present invention i.e. Hydroxycurcumin complex (HCC) as obtained in example 5 above and incubate the cells for 24 hrs.
3. After 24 hrs of incubation period, the supernatant was collected from all the wells and washed by cold PBS gently by centrifugation at 1,000×g for 5 minutes.
4. The whole supernatant in the assay was collected and transferred to a clean tube, being sure to use the Culture Medium as a blank.
5. The tube was kept in Ice and experiment based on EZ Assay Nitric Oxide estimation Kit was conducted.

### Preparation of Standard Curve (Total Nitric Oxide)

320 μM sodium nitrate (NaNO₃) solution was prepared using 200 mM NaNO₃ (CCK061(E)). For this, 16 μl (CCK061(E)) was added to 9984 μl of cell culture grade water. Serially dilute 320 μM NaNO₃ in 1:1 ratio to obtain 160 μM, 80 μM, 40 μM, 20 μM, 10 μM, 5 μM solutions.

### Total Nitric Oxide Assay Protocol

100 μL of culture supernatants were collected and mixed with the Griess reagent system. Then the plates were incubated for 2 hrs at 37° C. in the 5% CO₂ incubator. The absorbance was measured at 580 nm in a Multimode Microplate Reader (ELX 800, BioTek, India). Nitrite concentration was determined from a sodium nitrite standard curve.

COLORIMETRIC CALCULATIONS: The average absorbance of each standard and sample was calculated. The absorbance value of the standard A (0 μM) was subtracted from itself and all other values (both standards and samples). This is the corrected absorbance.

The corrected absorbance values of each standard were plotted as a function of NaNO₃ concentration. The values of Total Nitric oxide (NO) for each sample were calculated from the standard curve using following formula:

$$Total NO inhibition study = [ ( Corrected Absorbance - \frac{( y - intercept ) )}{Slope} ]$$

### Results:

FIG. 1 is depicting the overlay bar graph showing the % total NO production observed in the untreated control, H₂O₂, H₂O₂ and the composition of the present invention (HCC) in various concentrations (6.25, 12.5, 25, 50 and 100 μg/ml) versus the standard control taken of Silymarin at 200 μM/ml.

The % NO production as observed is also tabulated in Table 2 as under:

TABLE 2
The % total NO production observed in the H₂O₂ alone
induced cells and H₂O₂ conjugated with the composition
of the present invention (HCC).

| Culture condition | % NO production |
|---|---|
| H2O2 alone | 100 |
| H2O2 + HCC 6.25 μg | 91.99 |
| H2O2 + HCC 12.5 μg | 77.16 |
| H2O2 + HCC 25 μg | 56.96 |
| H2O2 + HCC 50 μg | 48.06 |
| H2O2 + HCC 100 μg | 12.95 |
| H2O2 + Sylimarin-200 μM | 15.17 |

It was observed that the composition of the present invention i.e. Hydroxycurcumin complex (HCC) as obtained in Example 5, significantly inhibited the Total NO production in a dose dependent manner.

### Conclusion:

The obtained Total NO inhibition assay results revealed that the H₂O₂ alone induced cells, released high percentage of Total Nitric oxide production whereas the composition of the present invention (HCC) with different non-toxic concentrations in H₂O₂ induced model, effectively inhibited the Total NO production in a dose dependent manner and significantly inhibited the Total NO release in 100 μg/ml concentration similar to the Standard control, Silymarin with 200 μM/ml concentration respectively.

### Example 8

### In-Vitro Study to Assess the Efficacy of the Composition of the Present Invention by Conducting the Cytotoxicity Study on A549 Cell Lines by MTT Assay

### Background of the Study:

MTT assay is a colorimetric assay used for the determination of cell proliferation and cytotoxicity, based on reduction of the yellow colored water soluble tetrazolium dye MTT to formazan crystals. Mitochondrial lactate dehydrogenase produced by live cells reduces MTT to insoluble formazan crystals, which upon dissolution into an appropriate solvent exhibits purple color, the intensity of which is proportional to the number of viable cells and can be measured spectrophotometrically at 570 nm. (Alley et al and Mosamann et al).

### Cell Line and Culture Medium:

This work was carried out in A549-Human alveolar lung adenocarinoma cell lines purchased from NCCS, Pune, India and the Cell culture medium was DMEM-high Glucose-(#AL111, Himedia). The A549 cells were maintained in DMEM high glucose media supplemented with 10% FBS along with the 1% antibiotic-antimycotic solution in the atmosphere of 5% CO₂, 18-20% O₂ at 37° C. temperature in the CO₂ incubator and sub-cultured for every 2-3 days. Passage Number 35 was used for the current study.

### Assay Controls:

(i) Medium control (medium without cells)-Blank
(ii) Negative control (medium with cells but without the experimental drug/compound)
(iii) Positive control (medium with cells treated with 20 μM/ml of Cisplatin)

Note: Extracellular reducing components such as ascorbic acid, cholesterol, alpha-tocopherol, dithiothreitol present in the culture media may reduce the MTT to formazan. To account for this reduction, it is important to use the same medium in control as well as test wells.

### Procedure:

200 μl cell suspension was seeded in a 96-well plate at required cell density (20,000 cells per well), without the test agent. The cells were allowed to grow for about 24 hours. The cells were treated with desired concentrations of the samples (HCC composition as obtained in Example 5 taken at different concentrations, control and standard control) and the plates were incubated for 24 hrs at 37° C. in a 5% CO₂ atmosphere. After the incubation period, the plates were taken out from incubator, and spent media removed and MTT reagent was added to a final concentration of 0.5 mg/mL of total volume. The plates were wrapped with aluminium foil to avoid exposure to light. The plates were returned to the incubator and incubated for 3 hours. (Note: Incubation time varies for different cell lines. Within one experiment, incubation time should be kept constant while making comparisons.). After the incubation period the MTT reagent was removed and then 100 μl of solubilisation solution (DMSO) was added while gentle stirring in a gyratory shaker to enhance dissolution. Occasionally, pipetting up and down may be required to completely dissolve the MTT formazan crystals especially in dense cultures. The absorbance was measured on a spectrophotometer or an ELISA reader at 570 nm wavelength.

% Cell viability is calculated using below formula:

% cell viability=[Mean abs of treated cells/Mean abs of Untreated cells]×100

The IC₅₀ value was determined by using linear regression equation i.e. Y=Mx+C.

Here, Y=50, M and C values were derived from the viability graph.

### Drug Concentrations Details:

In this study, the test compounds i.e. composition of the present invention HCC complex as obtained in Example 5 was evaluated to analyse the cytotoxicity effect on A549 cells. The concentrations of the samples taken (viz. standard, control and composition of the present invention (HCC) as obtained in Example 5) were used to treat the cells are as shown in Table 3 hereunder:

TABLE 3
Details of drug treatment to respective cell line used for the study

| Sl. No | Culture condition | Cell line | Concentrations treated to cells |
|---|---|---|---|
| 1 | Untreated | A549 | No treatment |
| 2 | Blank | — | Only Media without cells |
| 3 | Std control (Cisplatin) | A549 | 20 μM/mL |
| 4 | HCC | A549 | 6 (12.5, 25, 50, 100, 200, 400 μg/ml) |

### Results:

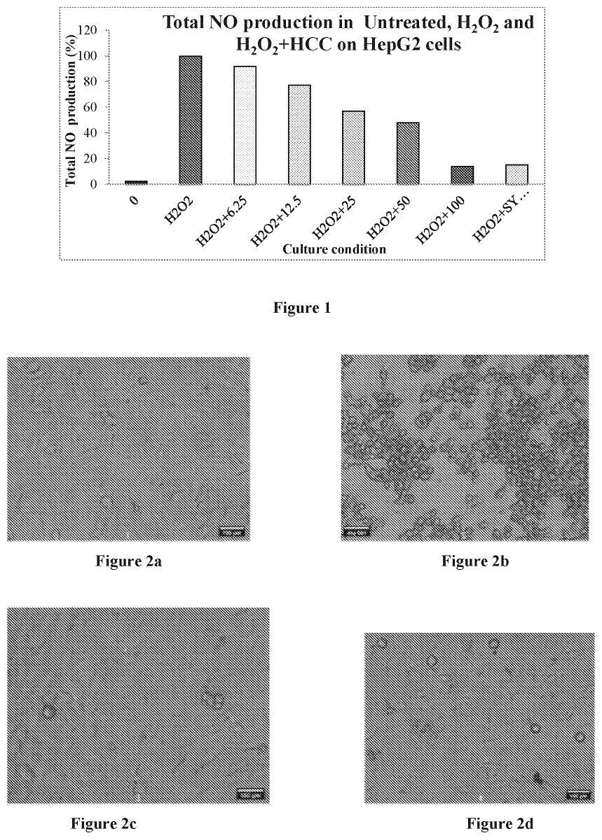

FIGS. 2a, 2b, 2c, 2d, 2e, 2f, 2g and 2h show the cytotoxicity effects of different concentrations of the composition of the present invention (Hydroxycurcumin complex (HCC)) along with controls like Untreated and Standard control after the incubation period of 24 hours. 2a-Untreated, 2b-Cisplatin with 20 μM, 2c through to 2h-HCC with 12.5, 25, 50, 100, 200 and 400 μg/ml.

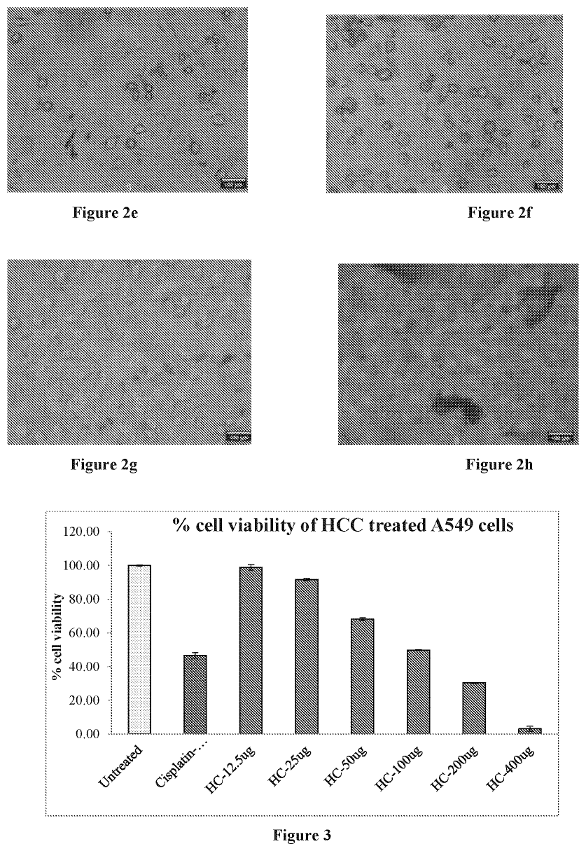

Green arrow in the FIGS. 2a through to 2h indicates healthy cells and Red arrow represents damaged cells with condensed or shrinked morphology of A549 cells. FIG. 3 is depicting the overlay bar graph showing the precent cell viability values of A549 cells treated with different concentrations of the composition of the present invention (HCC), vs. controls and standard control after the treatment period of 24 hrs. Table 4 is showing the % cell viability values of the composition of the present invention (HCC) treated on A549 cells after the treatment period of 24 hrs. It can be seen from Table 4 that the composition of the present invention i.e. hydroxycurcumin complex (HCC) was effectively cytotoxic in nature on A549 cells with low IC₅₀ concentration of 91 μg/ml after the incubation period of 24 hours. Cisplatin with 20 μM was used as a standard control for the study which showed 47% cell viability. The composition of the present invention i.e. Hydroxycurcumin complex (HCC) showed anti-lung cancer activity concentration dependent manner with decreased percentage cell viability and increased percentage of inhibition of cell growth. Different concentrations of HCC viz., 12.5, 25, 50, 100, 200 and 400 μg/ml showed the % cell viability values of 99%, 68%, 50%, 30% and 3% respectively.

TABLE 4
% cell viability values of the composition of the present
invention i.e. hydroxycurcumin complex (HCC) treated
on A549 cells after the treatment period of 24 hrs.

| Culture condition | % Cell viability | |
|---|---|---|
| Untreated | 100.00 | IC50 concentration = |
| Cisplatin-20 μM | 46.61 | 90.67 ug/ml |
| HCC-12.5 μg | 98.85 | |
| HCC-25 μg | 91.63 | |
| HCC-50 μg | 68.11 | |
| HCC-100 μg | 49.83 | |
| HCC-200 μg | 30.32 | |
| HCC-400 μg | 3.09 | |

### Conclusion:

The MTT assay exhibited that the composition of the present invention (HCC) was effectively cytotoxic in nature on A549 cells with low IC₅₀ concentration of 91 μg/ml after the incubation period of 24 hours. The composition of the present invention (HCC) exhibited anti-lung cancer activity.

### Example 9

In-Vivo Studies—Acute Oral Toxicity study in Wistar Rats

OBJECTIVE: The objective of this study is to assess the toxicity/safety of the composition of the present invention by acute oral administration in adult healthy female wistar albino rats.

### Materials and Methods

### Animals:

Species & strain: Rat & Albino Wistar
Sex: Female
Body weight: 80-200 gms
Diet: Normal diet

Animal Welfare: Animal experiment was conducted in accordance with the guidelines of committee for the purpose of control and supervision of experiments on animals (CPCSEA Registration Number-1 803/PO/RcBi/S/20 1 5/CPC SEA).

Animal Housing Conditions: Animals were housed under temperature of 22±3° C., relative humidity 30-70%, 12 hour light and 12 hour dark cycle. Animal was housed in a standard polypropylene cage with stainless steel top grill having facilities for food and water. Sterile Corncob (Source: Hylasco Biotechnology, Hyderabad) was used as bedding material and changed every day.

Feed and Water: Normal chow diet (Purina lab diet 5L79 Rat and Mouse 18%) (PMI Nutrition International) was provided to all the animals throughout the experiment. Fresh water was provided ad libitum. Animals were provided access to fresh, potable, uncontaminated drinking water. Periodic monitoring of microbial contamination of water was done. Drinking water bottles and their tubes was examined routinely to ensure their proper operation.

### Methods:

The present study was conducted in female Wistar rats by using OECD 423 guidelines. In the acute toxicity study, rats were administered a single dose of 2000 mg/kg orally and then observed individually for the first four hours, then over a period of 24 hours and once daily for 14 days. General behavior, adverse effects and mortality were observed throughout the experimental period. Body weights were recorded on test day 0 (prior to administration), day 3, day 7 and day 14. All the animals were necropsied and examined macroscopically.

Formulation: The formulation was prepared with the test item i.e. the composition of the present invention i.e. Hydroxycurcumin complex (HCC) of Example 5. The test item formulation was prepared daily. Required quantity of test item i.e. the composition of the present invention i.e. hydroxycurcumin complex (HCC) was taken in a mortar and pestle, and it was triturated and adequate quantity of vehicle was added, mixed well and transferred to a volumetric flask. Additional quantity of vehicle was added to the beaker, rinsed and transferred to the volumetric flask. Required volume was made up by adding sufficient quantity of vehicle in the volumetric flask, mixed well and transferred to the labeled beakers with magnetic beads. Homogeneity of the test item formulations during dose administration was maintained by continuous stirring using a magnetic stirrer. The amount of the test item and volume of the formulations prepared varied depending on the requirement and/or body weights of the animals. The exact amount of the test item, volume of the formulation prepared and volume of administration were recorded in the raw data.

Dose Administration: The test substance formulation i.e. the composition of the present invention i.e. hydroxycurcumin complex (HCC) was administered only once by oral route at the dose level of 2000 mg/kg bodyweight of an animal. The dose volume administered to each rat was 10 ml/kg/day. The dose volume was calculated for individual animals on the day of the treatment based on bodyweight.

Observations: Following oral administration of water and the composition of the present invention of Example 5, the mice were observed for mortality and morbidity, visible clinical signs including changes in skin, fur, eyes and mucous membranes and also respiratory, circulatory and behavioral pattern. Attention was also given to tremors, convulsions, salivation, diarrhea, lethargy and coma. Individual body weights were recorded at receipt, on the day of randomization, on the first day of treatment before dosing (day 0), then day 3, day 7 and day 14. The change in body weight for all animals were calculated and reported along with the body weight data.

Termination: At the end of the treatment period (Day 15), all the animals were sacrificed by using Isoflurane and subjected to gross pathological examination.

All data including body weight and clinical symptoms were statistically analyzed using Graph-Pad Prism Software, version 5.01. All values were expressed as mean±SEM. The significant difference between the treatment and control group was estimated using one-way ANNOVA with Dunnett's test. All results of the statistical analysis were summarized in separate tables. In any case the values were considered statistically significant at P<0.05.

RESULTS: The following conclusions were derived from oral administration of the composition of the present invention (HCC) at doses of 2000 mg/kg bodyweight of an animal.
1. Mortality or Morbidity was not observed in test substance treated animals throughout the experimental period (Refer Table 1).
2. Clinical Signs-All the animals were observed to be normal throughout the experimental period.
3. Body Weight and body Weight Changes-During the study period the test substance treated animals body weights were significantly increased when compared to day 0 bodyweight of animals (Refer Table 5).
4. Gross pathology—The gross pathological examination of test substance treated animals was found to be normal.

TABLE 5
Body weight of rats during the study

| | | Treatment | | | |
|---|---|---|---|---|---|
| | | Before | After | | |
| Animal ID | Dose | Day 0 | Day 3 | Day 7 | Day 14 |
| RA 01 | 2000 mg/kg | 183.6211.99 | 189.54 ± 2.12 | 195.03 ± 2.94 | 204.69 ± 2.90 |
| RA 02 | | | | | |
| RA 03 | | | | | |
| RA 04 | Limit test | 184.50 + 0.51 | 190.53 + 0.60 | 196.06 ± 0.71 | 209.88 ± 1.52 |
| RA 05 | (2000 mg/kg) | | | | |
| RA 06 | | | | | |
| RA 07 | | | | | |
| RA 08 | | | | | |
| RA 09 | | | | | |

*Values expressed as Mean ± SEM

CONCLUSION: All the surviving animals had gained body weight by day 14 as compared to day 0. No abnormalities were detected for the animals necropsied at terminal sacrifice. The limit doses of 2000 mg/kg did not cause any mortality or signs of toxicity in the rats tested during the observation period. As per OECD 423, if there is no lethality >2000 mg/kg body weight, then the dose falls under category 5. Based on the results, the median lethal dose of test substance in female rats after single oral dose 2000 mg/kg body weight and is classified as Category 5 and Safe.

While particular embodiments of the composition of present invention comprising therapeutically effective phytoingredients have been illustrated and described, it would be obvious to those skilled in the art that various other changes and modifications can be made within departing from the spirit and scope of the invention. It is thereof intended to cover in the appended claims such changes and modifications that are within the scope of the invention.

### REFERENCES

1. Velayudhan, K. C., Muralidharan, V. K., Amalraj, V. A., Gautam, P. L., & Mandal, S. K. (1999). Curcuma genetic resources (Scientific monograph no. 4, pp. 149). New Delhi: National Bureau of Plant Genetic Resources.
2. Watt, G. A. (1972). Dictionary of the economic products of India 1908 (p. 689). Delhi: Periodical Experts.
3. Nahar, L.; Sarker, S. D. Phytochemistry of the genus Curcuma. In Turmeric: the genus Curcuma, Ravindran, P. N.; Nirmal Babu, K.; Sivaraman, K., Eds. CRC Press: Boca Raton, 2007; pp 71-106.
4. MTT Cell Proliferation Assay Instruction Guide—ATCC, VA, USA www.atcc.org
5. Gerlier D., and N. Thomasset. J. Immunol. Methods 94:57-63, 1986. Alley, M. C., et al.
6. Cancer Res. 48: 589-601, 1988. Mosmann, T. J. Immunol. Methods 65:55-63, 1983.
7. Alley, M. C., Scudiere, D. A., Monks, A., Czerwinski, M., Shoemaker, R. II., and Boyd, M. R. Validation of an automated microculture tetrazolium assay (MTA) to assess growth and drug sensitivity of human tumor cell lines. Proc. Am. Assoc. Cancer Res., 27: 389, 1986
8. http://himedialabs.com/TD/CCK003.pdf
9. Mosmann T. Rapid colorimetric assay for cellular growth and survival: application to proliferation and cytotoxicity assays. J Immunol Methods. 1983; 65:55-63.

1. A composition comprising therapeutically effective phytoingredients obtained from the Curcumin Removed Turmeric Oil (CRTO) wherein the composition comprises Bisdem ethoxy curcumin (Hydroxycurcumin) in the range of 6-27%, Demethoxycurcumin is in the range of 9-13% and Curcumin in the range of 2-4% and other phenolic compounds.

2. The composition as claimed in claim 1 wherein the composition is effective in protecting the liver against oxidative stress.

3. The composition as claimed in claim 1 wherein the composition is effective in showing anti-cancer activity.

4. The composition as claimed in claim 1 wherein the composition is formulated into suitable dosage forms selected from a group comprising of powder, paste, tablets, syrups and/or capsules, softgels or any suitable formulation.

5. A process for preparing the composition as claimed in claim 1 wherein the process comprises the steps of:
a) removing the oil content of the Curcumin Removed Turmeric Oil (CRTO) by mixing the CRTO with a non-polar solvent wherein the ratio of the amounts of CRTO and the solvent used is 1:3 to 1:6 weight by volume and heating this mixture at a temperature of 35 to 60° C., for approximately 30 to 120 minutes;
b) filtering the extract and repeating the process 3 more times to effectively remove oil content from the CRTO and obtain a left-over material;
c) pooling all the four extractives and concentrating at a temperature of 40-55° C. in a rotary evaporator to obtain crude turmeric oil wherein the turmeric oil isolated contains not less than 60% of total turmerones including a, P and ar-turmerones;
d) adding a non-polar solvent to the left over material or spent and heating the solution at a temperature of 50-90° C., preferably 60-70° C., wherein the ratio of left over material and non-polar solvent is 1:3 to 1:6 weight by volume and the extraction time is 1 hour;
e) Filtering the extract through Whatman's filter paper;
f) Repeating the above process 7 more times till complete recovery of curcuminoids and pooling all the extractives and concentrating at a temperature of 50-80° C., preferably 60-70° C.;
g) reducing the volume of the extract thus obtained to 1:1 ratio on input and cooling to a temperature of 10-15° C. for 8 hours, obtaining the crystals, filtering and drying in an oven at a temperature of 50-90° C. for 8 hours to obtain curcuminoids powder;
h) drying the spent or left-over material at a temperature of 80° C. under vacuum to remove the solvent and cooling to room temperature, to obtain a reddish brown powder which is the composition comprising therapeutically effective phytoingredients;
i) analysing the composition by HPLC for its curcuminoids content.

6. The process as claimed in claim 5 wherein the temperature of heating the mixture of CRTO and non-polar solvent in step a) is preferably 40-55° C. and wherein the mixture is heated for preferably 45 to 60 minutes.

7. The process as claimed in claim 5 wherein the non-polar solvent in step a) is selected from hexane, toluene, heptane, pentane, petroleum ether, diethyl ether or any other suitable solvent.

8. The process as claimed in claim 5 wherein the non-polar solvent taken in step d) may be selected from ethyl acetate, diethyl ether, toluene, heptane, methylene di-chloride or any other suitable non-polar solvent.

9. The process as claimed in claim 5 wherein the curcuminoids recovered in step g) are 95% by weight.

10. A process for preparing the composition as claimed in claim 1 wherein the process comprises the steps of
a) removing the oil content of the Curcumin Removed Turmeric Oil by mixing the CRTO with a non-polar solvent wherein the ratio of the amounts of CRTO and the solvent used is 1:3 to 1:6 weight by volume;
b) heating this mixture at a temperature of 40-45° C. for approximately 45 to 60 minutes;
c) filtering the extract after 45 to 60 minutes and repeating the process 3 more times to effectively remove oil content from the CRTO and obtain a left-over material;
d) extracting the left over material in a polar solvent wherein the ratio of left over material and the polar solvent is 1:4 to 1:6 weight by volume;
e) taking a measured quantity of de-mineralized water in a round bottom flask and slowly adding the methanol extract to precipitate curcuminoids, filter, and dry the precipitate at a temperature of 60-85° C.;
f) obtaining the dried powder which is then analyzed by HPLC for its curcuminoids content.

11. The process as claimed in claim 10 wherein the non-polar solvent taken in step a) is selected from hexane, petroleum ether, heptane, pentane, toluene, diethyl ether or any other suitable solvent and wherein the polar solvent taken in step e) is selected from methanol, ethanol, iso-propyl alcohol or any other suitable solvent.
